SEQUENCE LISTING (18 sequences)

SEQ ID NO: 1 - DNA, 22 bp, Artificial Sequence
  primer Chi600f
ggcatcaacgacagcntnaaag

SEQ ID NO: 2 - DNA, 22 bp, Artificial Sequence
  primer Chi1200r
gtcntagctcatcaggaagatg

SEQ ID NO: 3 - DNA, 579 bp, Aeromonas hydrophila
ggcatcaacgacagcctcaaagagatctcaggcagtttcgaggcgctgcaacgctcctgcgccgggcgcgaagacttcaaggtctccatccacgatccctgggccgccatccagatggggcagggcaatctcaccgcctatgacgagccctacaagggcaacttcggcaacctgatggcgctcaagaaggcctatccggacctgaaaattctcccctccatcggcggttggaccctctccgaccccttcttcttcttcggtgacaagaccaagcgcgacaccttcgtcgcctcggtgaaggagtacctgcagacctggaaattcttcgacggggtggacatcgactgggagttcccgggcggtctgggggccaaccccaacctcggcagcgcctccgatggcgagacctatgtgcccctgatgaaggagttgcgcgccatgctcgacgagctgagcgcagagacgggtcgcacctacgagctcacctccgccatcagcgccggcggtgacaagattgccaaggtggactatcgcgccgcccagcaatacatgaatcacatcttcctgatgagctaagaa

SEQ ID NO: 4 - DNA, 579 bp, Pantoea glomerans
ggcatcaacgacagcgttaaagagatctccggcagcttcgaggcgctgcagcgctcctgtgccggccgcgaggacttcaaggtctccatccacgatccctgggccgccatccagatggggcagggcaatctcaccgcctatgacgaaccctacaagggcaacttcggcaacctgatggcgctcaagaaggcctatccggacctgaagatcctcccctccatcggtggctggaccccctccgatcccttcttcttcttcggtgacaagaccaaacgcgacaccttcgtcgcctcggtgaaggagtatctgcaaacctggaaattcttcgacggggtggacatcgactgggaattcccgggtggcctgggggccaaccccaacctcggcagcgcctccgacggcgaaacctatgtgctgctgatgaaggagctgcgcgccatgctcgacgaactgagcgcagagaccggccgcacctacgagctcacctccgccatcagcgctggcggtgacaagattgccaaggtggactatcgcgccgcccagcaatacatgaatcacatcttcctgatgagctaagac

SEQ ID NO: 5 - DNA, 579 bp, Aeromonas punctata
ggcatcaacgacagctttaaagagatcgagggcagcttccaggcgctgcagcgttcctgccagggccgtgaagacttcaaagtgtcgatccacgatcctttcgcggcgctgcagaaaggccaaaaaggcgtgaccgcctgggacgacccctacaagggcaacttcggccagctgatggcgctgaaacaggcgcggcccgacctgaaaatcctgccgtcgatcggcggctggaccctgtccgacccgttcttcttcatgggcgacaaggcgaagcgcgaccgtttcgtcggttcggtgaaggagttcctgcagacctggaaattctttgacggcgtggacatcgactgggaattcccgggcggccagggcgccaacccgaaactgggcagcgcgcaggatggggcggcctacgtgcaactgatgaaagagctgcgggcgatgctggatcagctgtcggcggaaaccggccgtaagtatgagctgacctccgccatcagcgccggcaaagacaaaatcgacaaggtggactacaacaccgcgcagaactcgatggaccacatcttcctgatgagctacgac

SEQ ID NO: 6 - DNA, 562 bp, Artificial Sequence
  reassembly DNA clone-A1
cgacagcgttaaagagatctccggcagcttcgaggcgctgcagcgctcttgtgccggccgcgaggacttcaaggtctccatccacgatccctgggccgccatccagatggggcagggcaatctcaccgcctatgacgaaccctacaagggcaacttcggcaacctgatggcgctcaagaaggcctatccggacctgaagatcctcccctccatcggtggctggaccccctccgatcccttcttcttcttcggtgacaagaccaagcgcgacaccttcgtcgcctcggtgaaggagtacctgcagacctggaaattcttcgacggggtgacatcgactgggagttcccgggcggtctgggggccaaccccaacctcggcagcgcctccgatggcgagacctatgtgcccctgatgaaggagttgcgcgccatgctcgacgagctgagcgcagagacgggtcgcacctacgagctcacctccgccatcagcgccggcggtgacaagattgccaaggtggactatcgcgccgcccagcaatacatgaatcacatcttcctgatga

SEQ ID NO: 7 - DNA, 531 bp, Artificial Sequence
  reassembly DNA clone-A2
aggcgctgcaacgctcctgcgccgggcgcgaagacttcaaggtctccatccacgatccctgggccgccatccagatggggcagggcaatctcaccgcctatgacgaaccctacaagggcaacttcggcaacctgatggcgctcaagaaggcctatccggacctgaaaattctcccctccatcggcggttggaccctctccgaccccttcttcttcttcggtgacaagaccaaacgcgacaccttcgtcgcctcggtgaaggagtatctgcaaacctggaaattcttcgacggggtggacatcgactgggagttcccgggcggtctgggggccaaccccaacctcggcagcgcctccgatggcgagacctatgtgcccctgatgaaggagttgcgcgccatgctcgacgagctgagcgcagagacgggtcgcacctacgagctcacctccgccatcagcgccggcggtgacaagattgccaaggtggactatcgcgccgcccagcaatacatgaatcacatcttcctgatga

SEQ ID NO: 8 - DNA, 534 bp, Artificial Sequence
  reassembly DNA clone-B62
ggcagtttcgaggcgctgcaacgctcctgcgccgggcgcgaagacttcaaggtctccatacacgatccctgggccgccatccagatggggcagggcaatctcaccgcctatgacgaaccctacaagggcaacttcggcaacctgatggcgctcaagaaggcctatccggacctgaagatcctcccctccatcggtggctggaccccctccgatcccttcttcttcttcggtgacaagaccaaacgcggcaccttcgtcgcctcggtgaaggagtatctgcaaacctggaaattcttcgacggggtggacatcgactgggaattcccgggtggcctgggggccaaccccaacctcggcagcgcctccgacggcgaaacctatgtgctgctgatgaaggagctgcgcgccatgctcgacgaactgagcgcagagaccggccgcacctacgagctcacctccgccatcagcgctggcggtgacaagattgccaaggtggactatcgcgccgcccagcaatacatgaatcacatcttc

SEQ ID NO: 9 - DNA, 534 bp, Artificial Sequence
  reassembly DNA clone-B63
ggcagcttcgaggcgctgcagcgctcctgtgccggccgcgaggacttcaaggtctccatccacgatccctgggccgccatccagatggggcagggcaatctcaccgcctatgacgagccctacaagggcaacttcggcaacctgatggcgctcaagaaggcctatccggacctgaaaattctcccctccatcggtggctggaccccctccgatcccttcttcttcttcggtgacaagaccaaacgcgacaccttcgtcgcctcggtgaaggagtatctgcaaacctggaaattcttcgacggggtggacatcgactgggaattcccgggtggcctgggggccaaccccaacctcggcagcgcctccgacggcgaaacctatgtgctgctgatgaaggagctgcgcgccatgctcgacgaactgagcgcagagaccggccgcacctacgagctcacctccgccatcagcgctggcggtgacaagattgccaaggtggactatcgcgccgcccagcaatacatgaatcacatcttc

SEQ ID NO: 10 - DNA, 534 bp, Artificial Sequence
  reassembly DNA clone-B67
ggcagtttcgaggcgctgcaacgctcctgcgccgggcgcgaagacttcaaggtctccatccacgatccctgggccgccatccagatggggcagggcaatctcaccgcctatgacgaaccctacaagggcaacttcggcaacctgatggcgctcaagaaggcctatccggacctgaagatcctcccctccatcggtgactggaccccctccgatcccttcttcttcttcggtgacaagaccaaacgcgacaccttcgtcgcctcggtgaaggagtatctgcaaacctggaaattcttcgacggggtggacatcgactgggaattcccgggtggcctgggggccaaccccaacctcggcagcgcctccgacggcgaaacctatgtgctgctgatgaaggagctgcgcgccatgctcgacgaactgagcgcagagaccggccgcacctacgagctcacctccgccatcagcgctggcggtgacaagattgccaaggtggactatcgcgccgcccagcaatacatgaatcacatcttc

SEQ ID NO: 11 - DNA, 532 bp, Artificial Sequence
  reassembly DNA clone-B69
cagtttcgaggcgctgcaacgctcctgcgccgggcgcgaagacttcaaggtctccatccacgatccctgggccgccatccagatggggcagggcaatctcaccgcctatgacgaaccctacaagggcaacttcggcaacctgatggcgctcaagaaggcctatccggacctgaagatcctcccctccatcggtggctggaccccctccgatcccttcttcttcttcggtgacaagaccaaacgcgacaccttcgtcgcctcggtgaaggagtatctgcaaacctggaaattcttcgacggggtggacatcgactgggagttcccgggcggtctgggggccaaccccaacctcggcagcgcctccgatggcgagacctatgtgcccctgatgaaggagttgcgcgccatgctcgacgagctgagcgcagagacgggtcgcacctacgagctcacctccgccatcagcgccggcggtgacaagattgccaaggtggactatcgcgccgcccagcaatacatgaatcacatcttc

SEQ ID NO: 12 - DNA, 532 bp, Artificial Sequence
  reassembly DNA clone-B28
gcagtttcgaggcgctgcaacgctcctgcgccgggcgcgaggcttcaaggtctccatccacgatccctgggccgccatccagatggggcagggcaatctcaccgcctatgacgagccctacaagggcaacttcggcaacctgatggcgctcaagaaggcctatccggacctgaaaattctcccctccatcggcggttggaccctctccgaccccttcttcttcttcggtgacaagaccaagcgcgacaccttcgtcgcctcggtgaaggagtatctgcaaacctggaaattcttcgacggggtggacatcgactgggaattcccgggcggtctgggggccaaccccaacctcggcagcgcctccgatggcgagacctatgtgcccctgatgaaggagctgcgcgccatgctcgacgaactgagcgcagagaccggccgcacctacgagctcacctccgccatcagcgctggcggtgacaagattgccaaggtggactatcgcgccgcccagcaatacatgaatcacatcttc

SEQ ID NO: 13 - DNA, 535 bp, Artificial Sequence
  reassembly DNA clone-B29
gcagtttcgaggcgctgcaacgctcctgcgccgggcgcgaagacttcaaggtctccatccacgatccctgggccgccatccagatggggcagggcaatctcaccgcctatgacgagccctacaagggcaacttcggcaacctgatggcgctcaagaaggcctatccggacctgaagatcctcccctccatcggtggctggaccccctccgatcccttcttcttcttcggtgacaagaccaaacgcgacaccttcgtcgcctcggtgaaggagtatctgcaaacctggaaattcttcgacggggtggacatcgactgggagttcccgggcggtctgggggccaaccccaacctcggcagcgcctccgacggcgaaacctatgtgctgctgatgaaggagctgcgcgccatgctcgacgagctgagcgcagagacgggtcgcacctacgagctcacctccgccatcagcgccggcggtgacaagattgccaaggtggactatcgcgccgcccagcaacacatgaatcacatcttcct

SEQ ID NO: 14 - DNA, 532 bp, Artificial Sequence
  reassembly DNA clone-B32
cagcttcgaggcgctgcagcgctcctgtgccggccgcgaggacttcaaggtctccatccacgatccctgggccgccatccagatggggcagggcaatctcaccgcctatgacgaaccctacaagggcaacttcggcaacctgatggcgctcaagaaggcctatccggacctgaagatcctcccctccatcggtggctggaccccctccgatcccttcttcttcttcggtgacaagaccaaacgcgacaccttcgtcgcctcggtgaaggagtatctgcaaacctggaaattcttcgacggggtggacatcgactgggagttcccgggcggtctgggggccaaccccaacctcggcagcgcctccgatggcgagacctatgtgcccctgatgaaggagttgcgcgccatgctcgacgagctgagcgcagagacgggtcgcacctacgagctcacctccgccatcagcgccggcggtgacaagattgccaaggtggactatcgcgccgcccagcaatacatgaatcacatcttc

SEQ ID NO: 15 - DNA, 526 bp, Artificial Sequence
  reassembly DNA clone-B1
cagtttcgaggcgctgcaacgctcctgcgccgggcgcgaagacttcaaggtctccatccacgatccctgggccgccatccagatggggcagggcaatctcaccgcctatgacgagccctacaagggcaacttcggcaacctgatggcgctcaagaaggcctatccggacctgaaaattctcccctccatcggcggttggaccccctccgatcccttcttcttcttcggtgacaagaccaaacgcgacaccttcgtcgcctcggtgaaggagtatctgcaaacctggaaattcttcgacggggtggacatcgactgggaattcccgggtggcctgggggccaaccccaacctcggcagcgcctccgacggcgaaacctatgtgctgctgatgaaggagctgcgcgccatgctcgacgaactgagcgcagagaccggccgcacctacgagctcacctccgccatcagcgctggcggtgacaagattgccaaggtggactatcgcgccgcccagcaatacatgaatcac

SEQ ID NO: 16 - DNA, 532 bp, Artificial Sequence
  reassembly DNA clone-B5
cagtttcgaggcgctgcaacgctcctgcgccgggcgcgaggacttcaaggtctccatccacgatccctgggccgccatccaggtggggcagggcaatctcaccgcctatgacgaaccctacaagggcaacttcggcaacctgatggcgctcaagaaggcctatccggacctgaagatcctcccctccatcggtggctggaccccctccgatcccttcttcttcttcggtgacaagaccaaacgcgacaccttcgtcgcctcggtgaaggagtatctgcaaacctggaaattcttcgacggggtggacatcgactgggaattcccgggtggcctgggggccaaccccaacctcggcagcgcctccgacggcgaaacctatgtgctgctgatgaaggagctgcgcgccatgctcgacgaactgagcgcagagaccggccgcacctacgagctcacctccgccatcagcgctggcggtgacaagattgccaaggtggactatcgcgccgcccagcaatacatgaatcacatcttc

SEQ ID NO: 17 - DNA, 532 bp, Artificial Sequence
  reassembly DNA clone-B6
cagtttcgaggcgctgcagcgctcctgtgccggccgcgaggacttcaaggtctccatctacgatccctgggccgccatccagatggggcagggcaatctcaccgcctatgacgaaccctacaagggcaacttcggcaacctgatggcgctcaagaaggcctatccggacctgaagatcctcccctccatcggtggctggaccccctccgatcccttcttcttcttcggtgacaagaccaaacgcgacaccttcgtcgcctcggtgaaggagtatctgcaaacctggaaattcttcgacggggtggacatcgactgggaattcccgggtggcctgggggccaaccccaacctcggcagcgcctccgacggcgaaacctatgtgctgctgatgaaggagctgcgcgccatgctcgacgagctgagcgcagagacgggtcgcacctacgagctcacctccgccatcagcgccggcggtgacaagattgccaaggtggactatcgcgccgcccagcaatacatgaatcacatcttc

SEQ ID NO: 18 - DNA, 533 bp, Artificial Sequence
  reassembly DNA clone-B10
gcagcttccaggcgctgcagcgttcctgccagggccgtgaagacttcaaagtgtcgatccacgatcctttcgcggcgctgcagaaaggccaaaaaggcgtgaccgcctgggacgacccctacaagggcaacttcggccagctgatggcgctgaaacaggcgcggcccgacctgaaaatcctgccgtcgatcggcggctggaccctgtccgacccgttcttcttcatgggcgacaaggcgaagcgcgaccgtttcgtcggttcggtgaaggagttcctgcagacctggaaattctttgacggggtggacatcgactgggaattcccgggtggcctgggggccaaccccaacctcggcagcgcctccgacggcgaaacctatgtgctgctgatgaaggagctgcgcgccatgctcgacgaactgagcgcagagaccggccgcacctacgagctcacctccgccatcagcgctggcggtgacaagattgccaaggtggactatcgcgccgcccagcaatacatgaatcacatcttc

(19) United States
(12) Patent Application Publication
(10) Pub. No.: US 2006/0057567 A1
(43) Pub. Date: Mar. 16, 2006

(54) Method for constructing a chimeric dna library using a single strand speific dnase

(76) Inventor: Soon Gyu Hong, Kyungki-do (KR)

Correspondence Address: David A Einborn;Anderson Kill & Olick, 1251 Avenue of the Americas, New York, NY, 10020, US

(21) Appl. No.: 10/478,825

(22) PCT Filed: May 23, 2002
(86) PCT No.: PCT/KR02/01011

§ 371 Date: Nov. 21, 2003

(30) Foreign Application Priority Data
May 23, 2001 (KR) 2001/28442

(51) Int. Cl. C40B 40/08; C12P 19/34

(52) U.S. Cl. 435/6; 435/91.2

(57) ABSTRACT

The method for constructing a chimeric DNA library of the present invention uses matching regions of heterologous DNA sequences as internal primers and enables highly efficient DNA reassembly between heterologous DNAs having relatively low similarity. Therefore, the inventive method can be effectively used for developing a useful gene having better functional property through reassembly between various heterologous DNA sequences of enzyme-encoding gene, promoter, virus and so on.

### FIELD OF THE INVENTION

The present invention relates to a method for constructing a chimeric DNA library using complementary internal primers produced by treating partially hybridized single strands of heterologous DNA sequences with a single strand-specific DNase, and a directed evolution method using the chimeric DNA library to evolve DNA sequences.

### BACKGROUND OF THE INVENTION

Numerous enzymes are used in pharmaceutical and other industries and many efforts have been made to improve each enzyme's particular function. Many of such studies have relied on random mutation of the enzyme-encoding gene or protein engineering based on previously established relationship between enzyme structure and its function, but the results were only marginal. Recently, directed evolution has been employed as a means to develop a gene that has improved functional property.

The directed evolution is carried out first by constructing a mutant library by introducing mutation into various sites of protein-encoding genes. Many mutant libraries have been constructed by using several methods such as chemical mutagenesis, error-prone PCR, mutagenic PCR using random oligonucleotide, saturation mutagenesis, cassette mutagenesis, incremental truncation, homologous recombination and bacterial mutator strain. However, these methods have problems in that mutation tends to occur at a certain localized site, it is difficult to introduce mutations at various sites simultaneously, and they are time-consuming as well as labor intensive.

To overcome such problems, Stemmer has developed a DNA shuffling method which is capable of giving DNA libraries of increased variety through shuffling between randomly introduced mutations (Stemmer W. P. C., Nature 370:389-391, 1994). This method comprises: randomly cleaving two or more kinds of DNAs with DNase I and conducting reassembly using partially hybridized DNA fragments both as templates and primers. A DNA library prepared by this method contains various reassembled DNA fragments having random mutation and it can be screened by a proper selection procedure and subjected to another cycle of shuffling (U.S. Pat. No. 6,165,793; U.S. Pat. No. 5,811,238; U.S. Pat. No. 5,830,721; U.S. Pat. No. 5,834,252; U.S. Pat. No. 5,837,458). Stemmer has carried out directed evolution of β-lactamase using this method to improve its resistance to cefotaxim over 32,000-fold.

Also reported are methods comprising: stopping DNA synthesis by using UV, adduct or mutagen; generating various single strand DNA fragments of variable length; and conducting reassembly (U.S. Pat. No. 5,965,408), as well as so-called StEP method and RPR method (Zhao et al., Nature Biotechnol. 16:258˜261, 1998; Shao et al., Nucl. Acids Res. 26:681˜683, 1998).

However, the above methods are not effective in the reassembly of heterologous DNA fragments having low sequence similarity, and there has existed a need to develop an improved method for constructing a chimeric DNA library that can be advantageously used in directed evolution.

### SUMMARY OF THE INVENTION

Accordingly, it is a major object of the present invention to provide a method for constructing a novel chimeric DNA library from various heterologous DNA sequences, e.g., enzyme-encoding genes that can be used to enhance the production of industrially useful proteins, DNAs, or RNAs.

In accordance with one aspect of the present invention, there is provided a method for constructing a chimeric DNA library from heterologous DNA sequences, which comprises the steps of:
a) obtaining single strands of the heterologous DNA sequences and hybridizing them to obtain a partially hybridized DNA;
b) cleaving single strand regions of the partially hybridized DNA using a single strand-specific DNase to generate double strand DNA fragments;
c) denaturing the double strand DNA fragments to generate single strand oligonucleotides;
d) conducting a series of polymerase chain reactions (PCR) using the single strand oligonucleotides as internal primers and the single strands obtained in Step (a) as templates to obtain elongated reassembled DNAs; and
e) amplifying the reassembled DNA by PCR using terminal primers which have the nucleotide sequence complementary to those at the both ends of the heterologous DNAs to be reassembled.

### BRIEF DESCRIPTION OF THE DRAWINGS

The above and other objects and features of the present invention will become apparent from the following description of the invention, when taken in conjunction with the accompanying drawings which respectively show;

FIG. 1: a schematic diagram illustrating the inventive method for constructing a chimeric DNA library using a single stand-specific DNase;

FIG. 2: a detailed drawing of Step (3 and 4) of FIG. 1, wherein

(C), (F) or (J) is a DNA fragment of various length synthesized by using a heterologous DNA template and internal primers,

(D), (G), or (K) is a DNA fragment synthesized by using DNA fragment (C), (F) or (J) as a template and terminal primers,

(E) is an example of DNA reassembly using DNA fragment (B) as a template and DNA fragment (D) as a primer,

(H) or (I) is an example of DNA reassembly via cross-priming between intermediate DNA fragments that are synthesized by using DNA fragments (A) and (B) as templates and internal primers,

(L) is an example of DNA reassembly using a DNA fragment that has underwent reassembly once as a primer and an original DNA fragment (A) as a template.

FIGS. 3 and 4: the nucleotide sequence of Clone A1 obtained in Example 2 which was reassembled from the chitinase genes of Aeromonas hydrophila (KCTC 2358) and Pantoea agglomerans (KCTC 2578) by the inventive method and those of the wild-types;

FIGS. 5, 6, 7 and 8: nucleotide sequences of selected clones obtained in Example 3 which were reassembled from the chitinase genes of Aeromonas hydrophila (KCTC 2358) and Pantoea agglomerans (KCTC 2578) by the inventive method and those of the wild-types;

FIGS. 9, 10 and 11: nucleotide sequences of selected clones obtained in Example 4 which were reassembled from the chitinase genes of Aeromonas hydrophila (KCTC 2358) and Pantoea agglomerans (KCTC 2578) by the inventive method and those of the wild-types;

FIGS. 12, 13 and 14: nucleotide sequences of selected clones obtained in Example 5 which were reassembled from the chitinase genes of Aeromonas hydrophila (KCTC 2358) and Pantoea agglomerans (KCTC 2578) by the inventive method and those of the wild-types;

FIG. 15: the nucleotide sequence of a clone obtained in Example 6 which was reassembled from the chitinase genes of Pantoea agglomerans (KCTC 2578) and Aeromonas puctata (KCTC 2944) by the inventive method and those of the wild-types.

### DETAILED DESCRIPTION OF THE INVENTION

As used therein, the term “reassembly” means the formation of a new DNA sequence by replication reaction accompanying template switching, cross-priming, or mismatch priming among different DNA sequences, and a “chimeric DNA”, a DNA reassembled by cross-linking heterologous DNA fragments as above.

The term “internal primer” or “complementary internal primer” as used herein means a single-stranded oligonucleotide used for inducing reassembly between heterologous DNA sequences. It can be generated by the steps of: hybridizing single strand heterologous DNA sequences to obtain a partially hybridized DNA, cleaving single strand regions of the partially hybridized DNA using a single strand-specific DNase to generate double strand DNA fragments, and denaturing the double strand DNA fragments to generate single-stranded oligonucleotides. And an internal primer can also be produced by artificial synthesis, and used in PCR amplification employing a single strand DNA as a template. In case the DNA sequence of a certain gene is well-known, reassembly of the target gene can be easily accomplished by using a artificially synthesized complementary internal primer without going through the steps of partial hybridization, digestion of single strand DNA moieties and denaturation. In such a case, the reaction condition can be easily optimized by controlling the concentration of the internal primer.

The term “terminal primer” as used herein means an oligonucleotide which has a nucleotide sequence complementary to the terminal sequence of a target gene to be reassembled. It is used in a small quantity in the first stage PCR for generating an elongated target gene and in a large quantity in the second stage PCR.

The term “chimeric DNA library” as used herein means various DNA fragments generated when heterologous DNA sequences are reassembled, and the term is sometimes used to indicate a host cell line, e.g., E. coli, transformed with an expression vector bearing reassembled DNAs.

The term “directed evolution” as used herein means to enhance the functional property of a gene via mutagenesis, wherein the mutation is achieved by constructing a chimeric DNA library via a heterologous DNA reassembly. The directed evolution is a process to obtain a gene having improved functional property by selecting a specific target gene from the chimeric DNA library.

The term “heterologous DNA” as used herein means a DNA containing DNA fragments originating from different cells, different kinds of DNA fragments isolated from one cell, or DNA fragments having different sequences by introducing mutation into the same gene via error-prone PCR or other methods. Heterologous DNAs having the same kind of genes are preferably employed in directed evolution, but it is possible to use genes originating from same or different species which encode proteins having biologically similar activity. Further, to generate a new gene that encodes a heterologous protein showing complete different activity, it is possible to use a protein-encoding gene unrelated to the original aim. However, since the heterologous DNAs must have some matching sequence regions for generating internal primers as described above, it is preferable to use genes showing a sequence similarity of more than 50%.

The term “single strand (-specific) DNase” as used herein means a DNA-digesting enzyme which selectively acts on single strand polynucleotides or single strand regions of a partially hybridized polynucleotide.

The present invention provides a method for constructing a chimeric DNA library through reassembly between single strand heterologous DNA fragments having different sequences, which comprises the steps of:
a) obtaining single strands of the heterologous DNA sequences and hybridizing them to obtain a partially hybridized DNA;
b) cleaving single strand regions of the partially hybridized DNA using a single strand-specific DNase to generate double strand DNA fragments;
c) denaturing the double strand DNA fragments to generate single strand oligonucleotides;
d) conducting a series of polymerase chain reactions (PCR) using the single strand oligonucleotides as internal primers and the single strands obtained in Step (a) as templates to obtain elongated reassembled DNAs; and
e) amplifying the reassembled DNA by PCR using terminal primers which have the nucleotide sequence complementary to those at the both ends of the heterologous DNAs to be reassembled.

As mentioned above, the inventive method for constructing the chimeric DNA library depends on selecting and isolating matching complementary sequence of the single strand heterologous DNA sequences through the use of a single strand-specific DNase and using them as internal primers for PCR amplification. Therefore, the inventive method is characterized highly efficient reassembly between heterologous DNA sequences having low sequence similarity.

Hereinafter, the inventive chimeric DNA constructing method and the directed evolution method using the chimeric DNA library are described in detail.

Procedure 1: Preparation of Internal Primer

(Step 1) Hybridization of Heterologous Single Strand DNAs

To hybridize heterologous DNA sequences bearing target genes to be reassembled, heterologous DNA sequences may be mixed, denatured by heating at a high temperature, and hybridized by gradually lowering the temperature. It is also possible to use a rapid denaturation method and induce hybridization at a relatively high temperature. For example, heterologous DNA sequences may be denatured by heating at above 90° C. for 10 min, cooled by soaking on ice to fix the denaturation state, and hybridized at 50˜70° C., preferably about 65° C., for 1˜12 hours in the presence of a salt. Alternatively, hybridization of heterologous DNA sequences may be conducted by gradually cooling over a period of 2˜3 hours after being denatured at a high salt concentration.

Heterologous DNA sequences to be hybridized contain genes to be subjected to directed evolution. For instance, to obtain a chitinase having enhanced chitin-degrading activity, chitinase genes purified from various species of cells can be used as starting materials to construct a chimeric DNA library. Heterologous DNA sequences used in the present invention may be in the elongated or restriction enzyme-digested form. In case of using restriction enzyme-digested DNAs as starting materials, the hybridization reaction takes place more efficiently.

In a preferred embodiment of the present invention, DNA reassembly is performed using three species of chitinase genes purified from Aeromonas hydrophila (KCTC 2358), Pantoea agglomerans (KCTC 2578), and Aeromonas punctata (KCTC 2944). The chitinase genes of Aeromonas hydrophila and Pantoea agglomerans showing relatively high sequence similarity can be easily reassembled by the inventive method (Examples 2 to 5), while the chitinase genes of Pantoea agglomerans and Aeromonas punctata which have relatively low sequence similarity can also be reassembled (Example 6). Restriction enzyme-digested DNAs serve as excellent templates to increase the hybridization efficiency (Example 3).

(Step 2) Treatment with a Single Strand-Specific DNase

In Step (1), complementary sequence regions of heterolohous DNA sequences are hybridized to form double stranded region, and the non-matching sequence regions exist in the single strand form. By treating such a partially hybridized product with a single strand-specific DNase, it is possible to select and isolate the hybridized double strand DNA fragments. The single strand-specific DNase may be S1 nuclease or Mung Bean nuclease.

For example, in case of using S1 nuclease, it is preferred that the partially hybridized heterologous DNAs are reacted at a temperature ranging from 37 to 45° C. in a concentration ranging from 100 to 1,000 units of S1/ml to selectively cleave single stranded regions. The enzyme reaction is preferably conducted at about 45° C. at an S1 nuclease concentration of 1,000 units/ml.

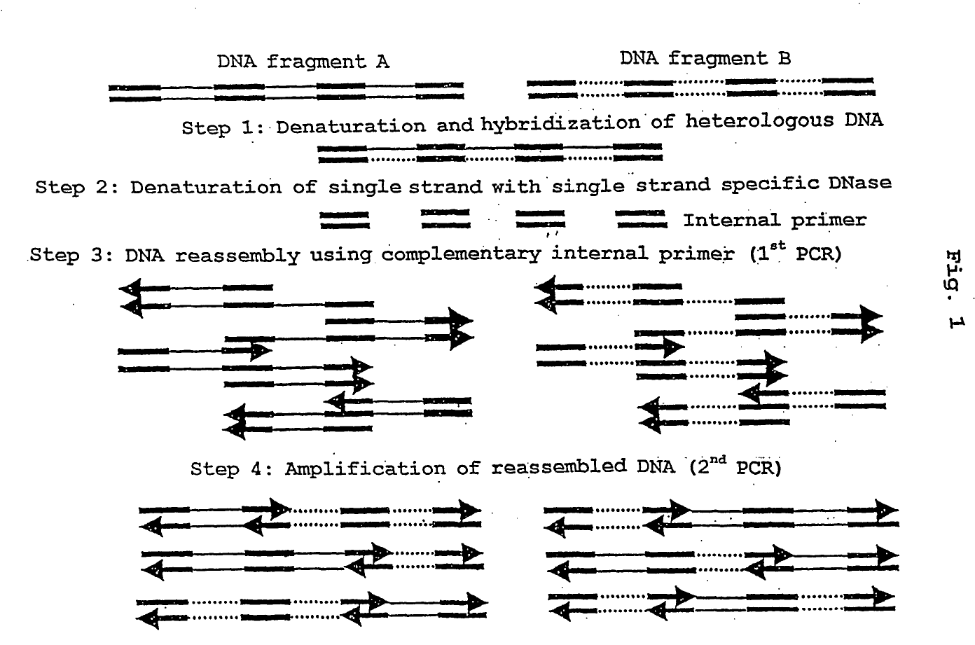

FIG. 1 depicts a schematic diagram illustrating the inventive method for constructing a chimeric DNA library. In FIG. 1, heavy solid lines represent conserved sequence regions of heterologous DNA sequences, and light solid lines and dotted lines, non-matching sequence regions.

(Step 3) Denaturation for Preparing Internal Primer

The double strand DNA fragments obtained in Step (2) are denatured to form single strand oligonucleotides by using any of the conventional methods. The single strand oligonucleotides obtained in this step can be used as internal primers in reassembly of target genes. Such internal primers may be artificially synthesized based on the conserved regions of heterologous DNA sequences.

Procedure 2: DNA reassembly Using Internal Primer (1^{st} PCR)

DNA reassembly is accomplished by PCR amplification using internal primers prepared in Procedure 1. In order to distinguish from the second stage PCR amplification (2^{nd} PCR) performed by adding a large quantity of terminal primers to amplify the reassembled DNA, this first stage procedure is designated 1^{st} PCR amplification.

1^{st} PCR amplification is performed using the heterologous DNAs as DNA templates and the internal primers generated above in the presence of a small quantity of added terminal primers. The template DNAs are the heterologous single strand DNA sequences used for generating the internal primers and it is preferable that both ends or one end of each template DNA are removed by treating with suitable restriction enzymes. When such enzyme-treated template DNAs are used, amplification thereof can be prevented during the second stage amplification conducted using a relative large amount of terminal primers, with a consequential selective enhancement of reassembled DNA amplification.

PCR amplification may be performed according to a conventional method, e.g., that involves a primary denaturation step at 94° C. for 3 min, 45 cycles of a denaturation step at 94° C. for 30 sec, an annealing step at 50° C. for 30 sec, an extension step at 72° C. for 5 sec, and a further extension step at 72° C. for 30 min.

If both ends of the heterologous DNA nucleotide sequences to be reassembled are conserved and present as a part of the internal primers, there is no need to add terminal primers during the 1^{st} PCR. Otherwise, terminal primers must be added in a small quantity, preferably in a concentration ranging from 0.02 to 0.1 pmole/50 μl. During the 1^{st} PCR, single stranded DNAs of various length are generated depending on the location of the internal primers attached to the template and such PCR products of single stranded DNAs act as secondary templates, eventually producing full-length reassembled DNA by the action of added terminal primers (see FIG. 1).

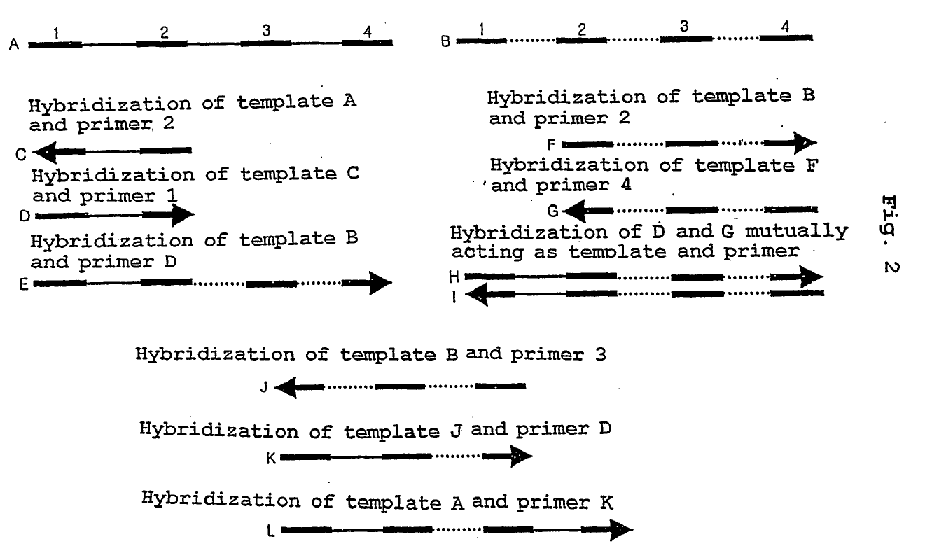

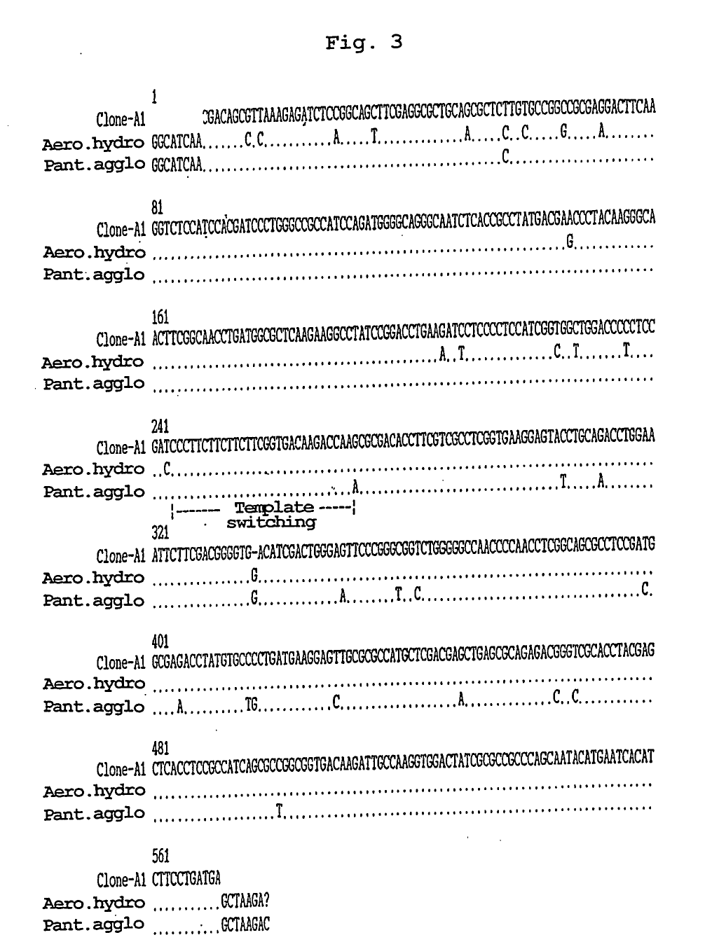

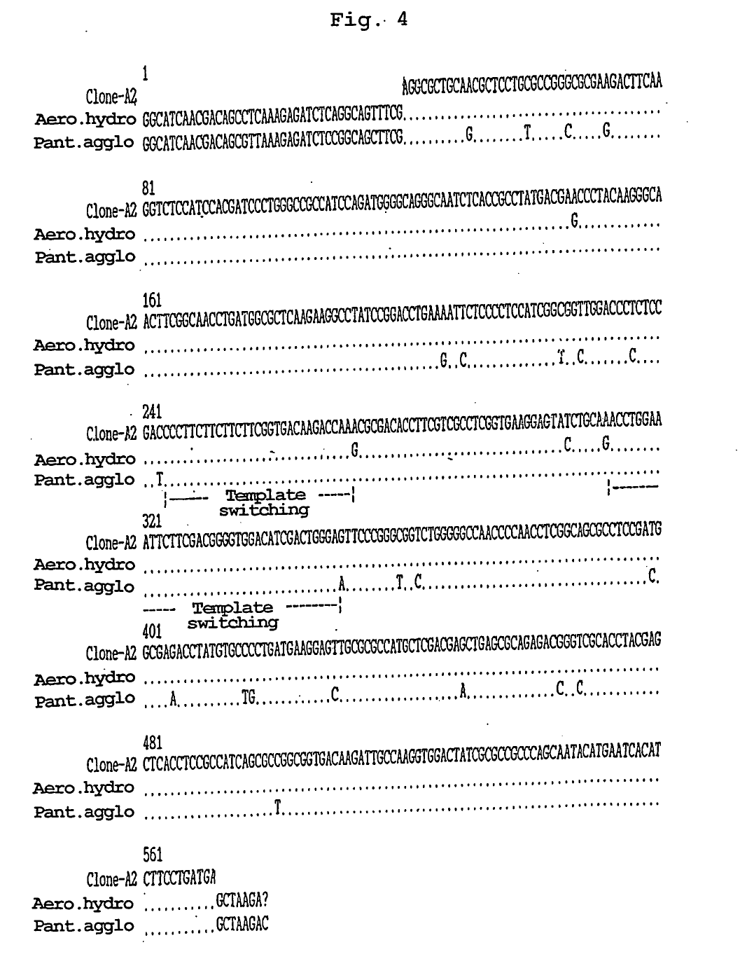

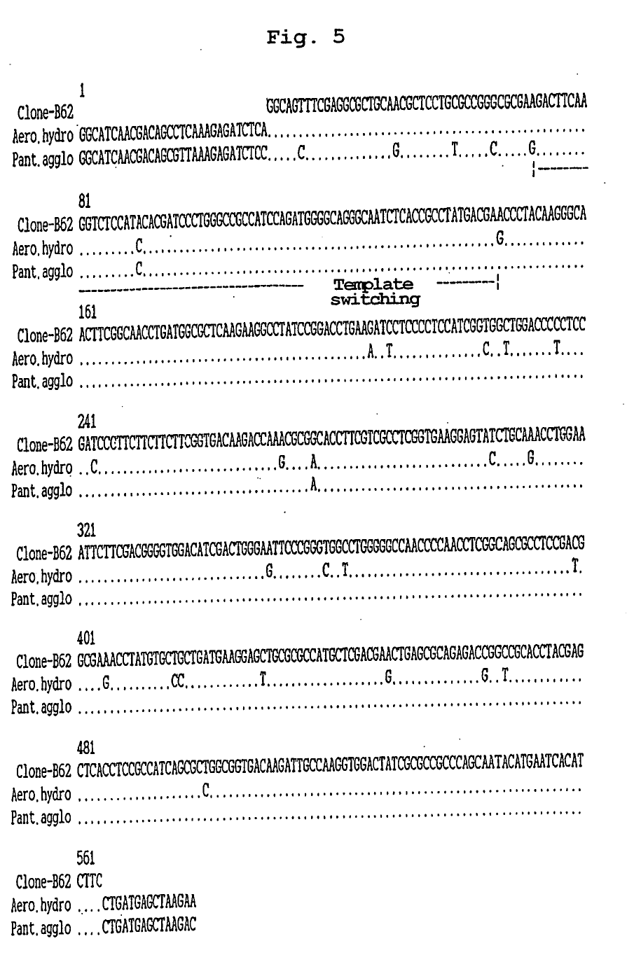

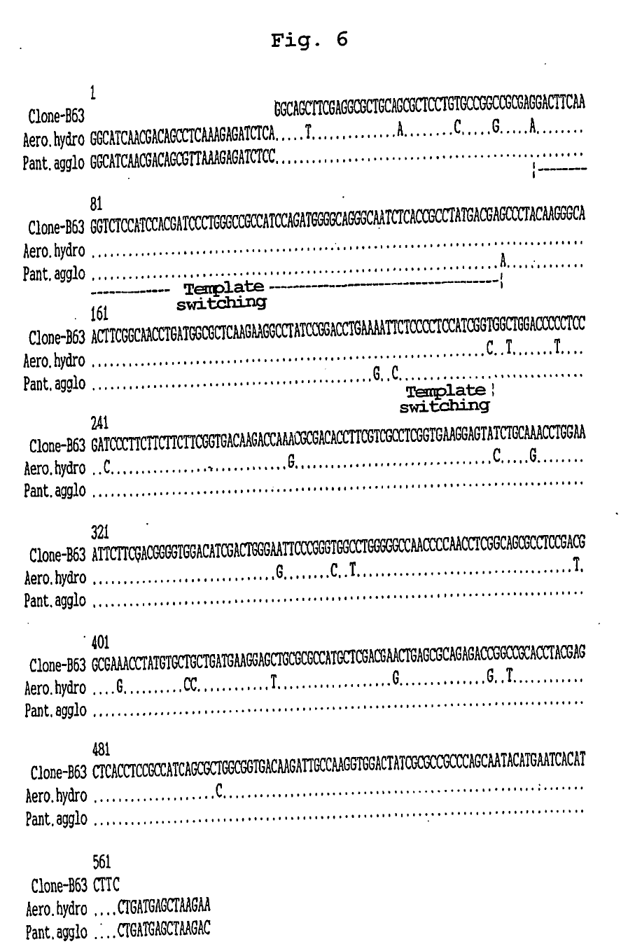

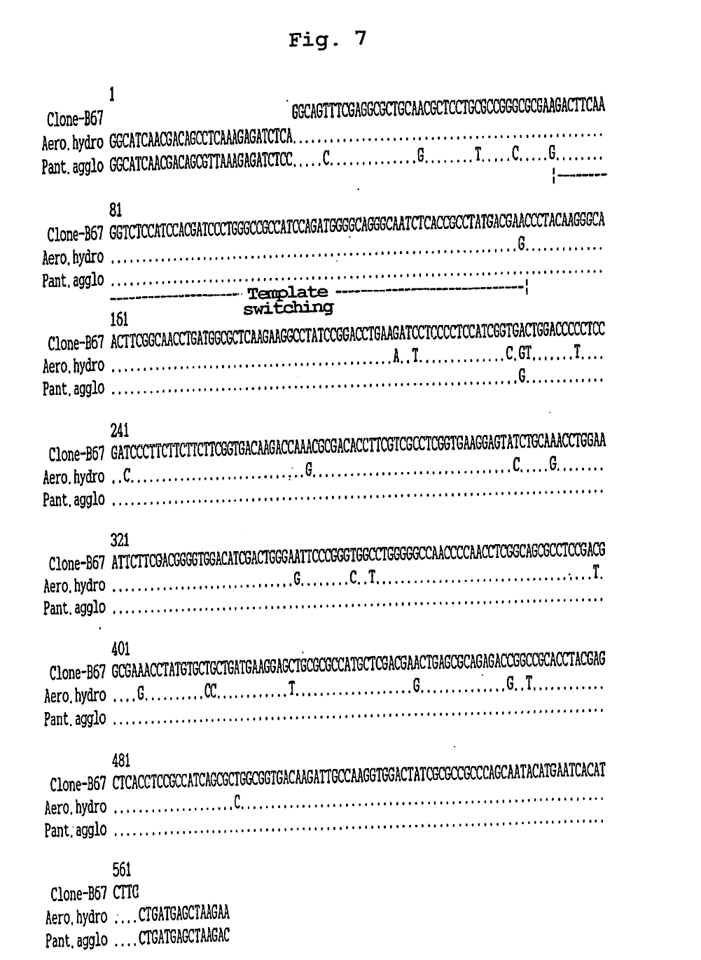

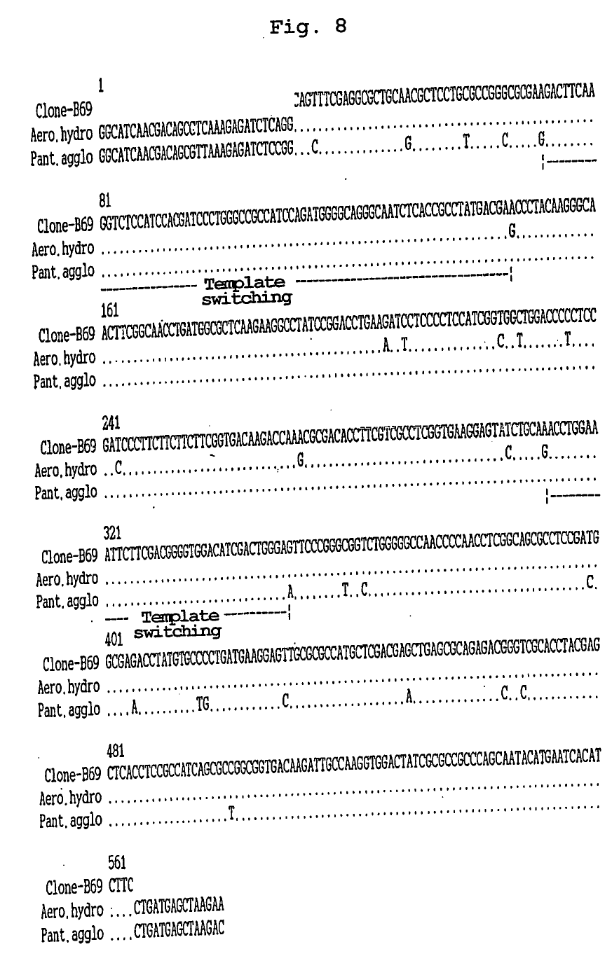

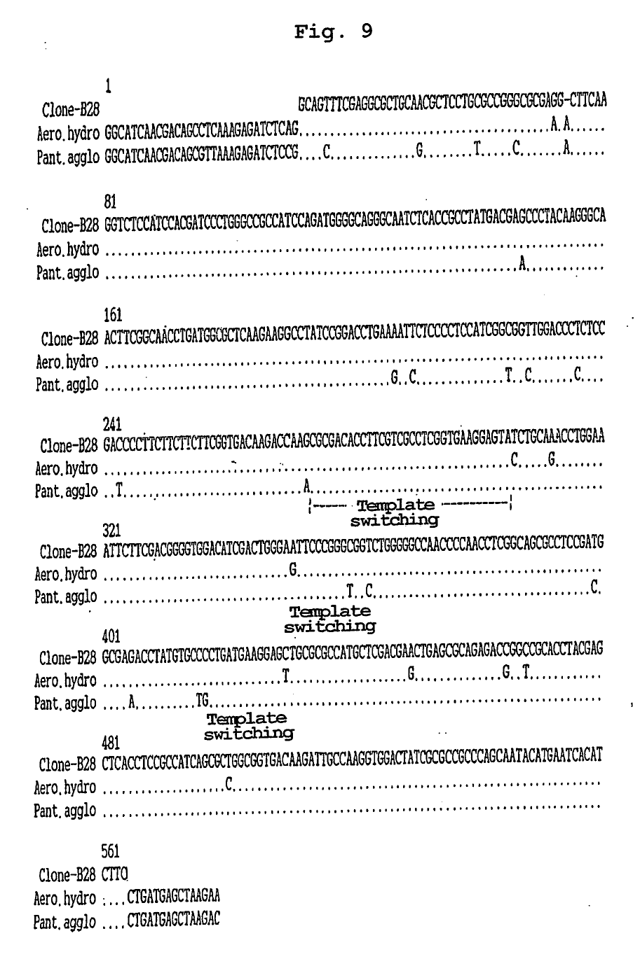

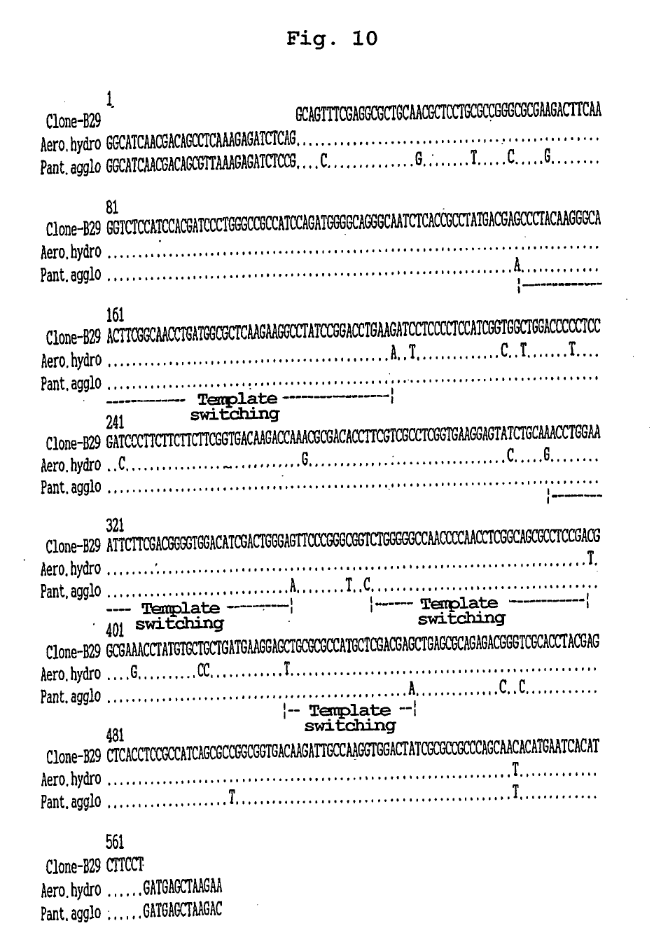

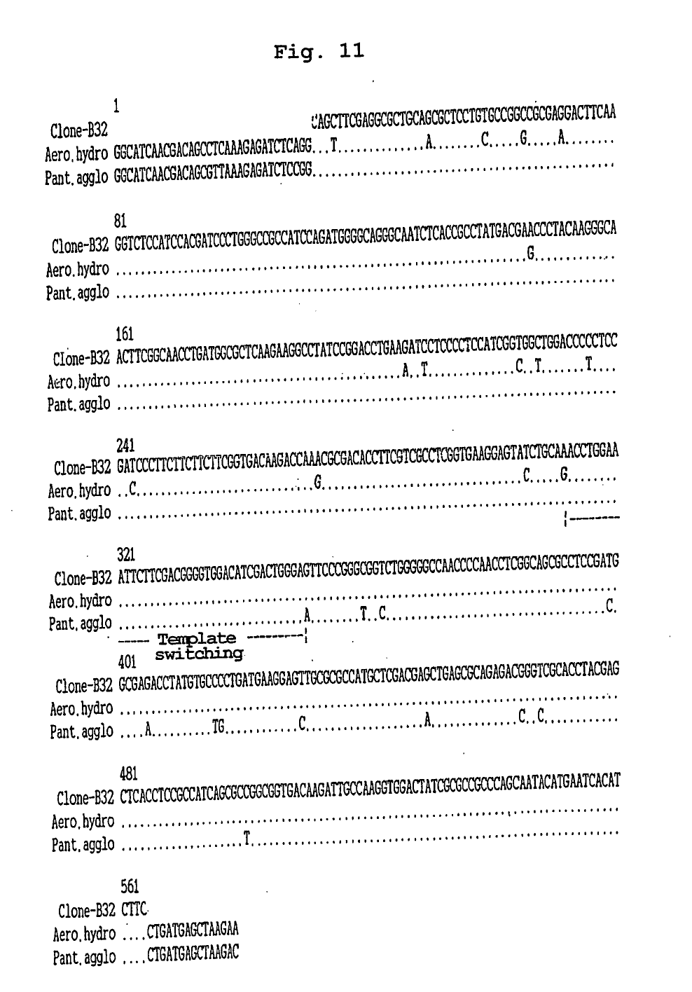

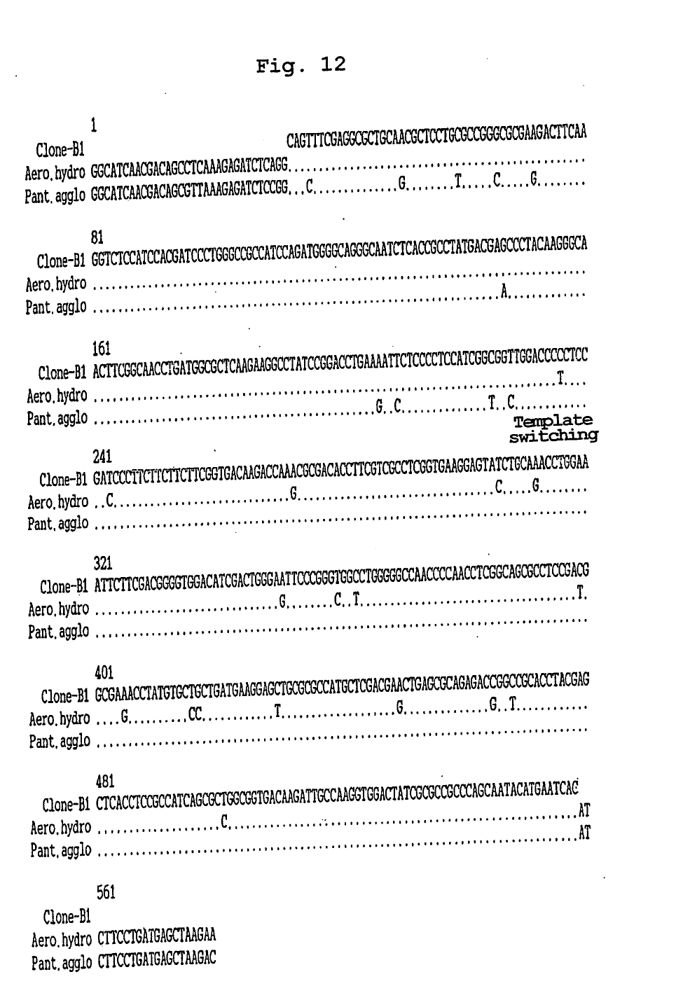

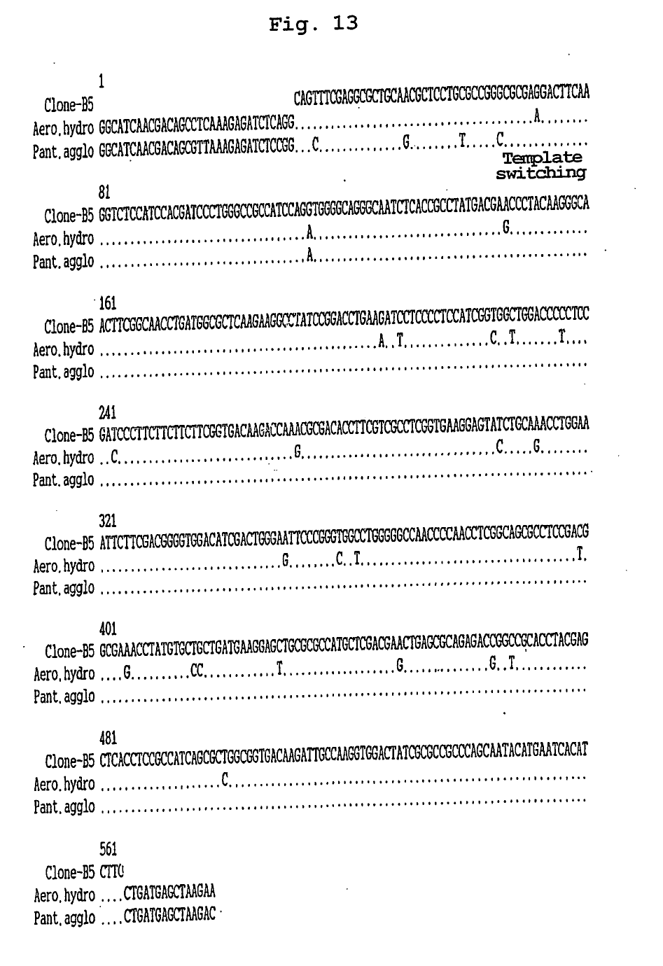

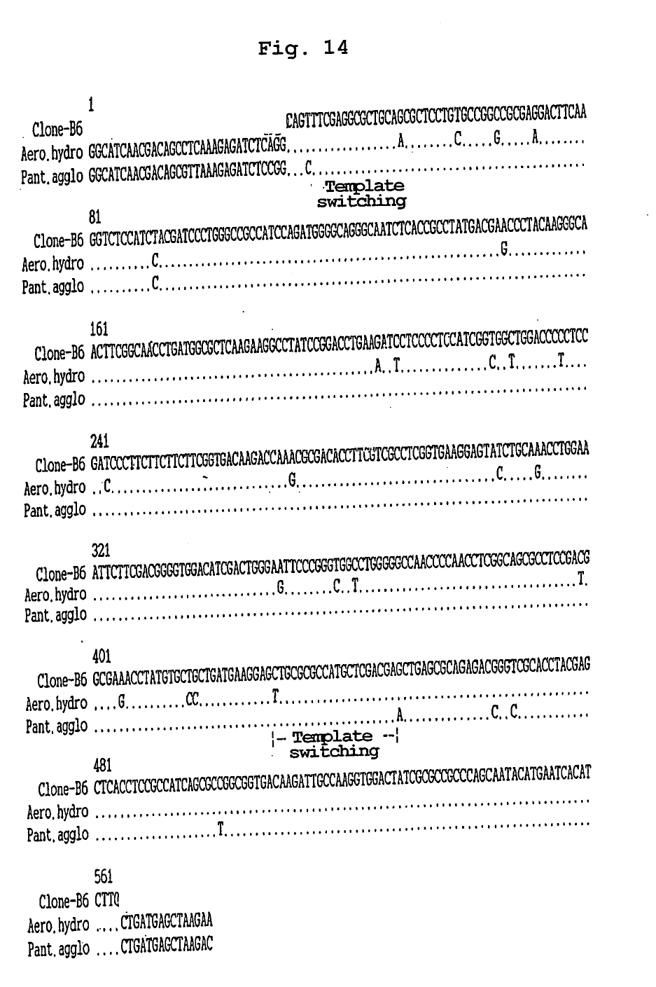

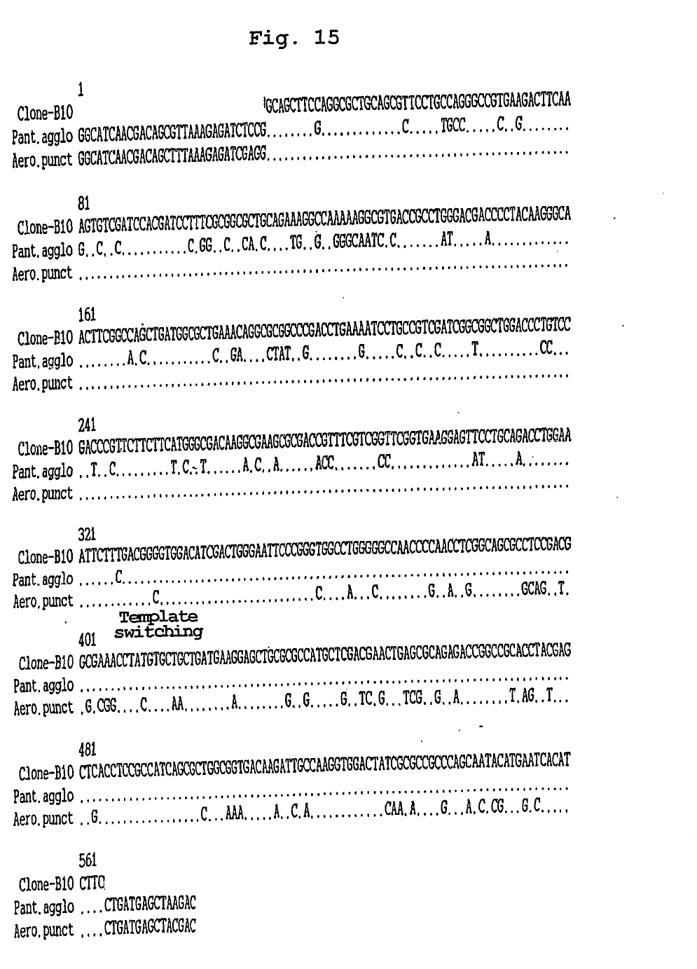

That is, as shown in FIG. 2, PCR products amplified by using heterologous DNA sequences as templates are elongated from the internal primers and they act simultaneously as primers and templates in further amplification reactions. The PCR products can also undergo template switching into the other DNA during the PCR amplification process of repeated denaturation and hybridization.

Through this procedure, all kinds of DNA reassembly can be generated. In addition, it is possible that additional reassembly can occur by cross-priming between reassembled DNA fragments containing the nucleotide sequences of terminal primers and even two or more cycles of reassembly can possibly take place by cross-priming between reassembled DNA fragments having no terminal primer nucleotide sequences.

Thus, the 1^{st} PCR involves at least two cycles of denaturation, hybridization and polymerization. Full-length reassembled DNA fragments are generated by cross-priming or template switching during this procedure, together with mutation sites, the number of which increases with the cycle number.

Procedure 3: Reassembled DNA Amplification Using a Large Quantity of Terminal Primers (2^{nd} PCR)

The full-length reassembled DNAs generated in the 1^{st} PCR reaction are amplified by conducting 2^{nd} PCR amplification in the presence of a large quantity of terminal primers. The reason why the 1^{st} and 2^{nd} PCR reactions are conducted separately is that original template DNA becomes predominant in the presence of a large amount of terminal primers which directly act on template DNAs, with a consequential decline in the reassembled DNA fragment.

To amplify the reassembled DNA fragments generated in the 1^{st} PCR amplification, it is preferable to use terminal primers at a concentration ranging from 20 to 25 pmole/50 μl, preferably about 25 pmole/50 μl.

The reassembled DNA fragments thus prepared may be introduced into an expression vector, which may in turn be used to transform microorganisms e.g., E. coli. The chimeric DNA library of the present invention may be applied to improve the functions of various genes, e.g., enzyme-encoding gene, promoter, virus gene, and so on via directed evolution to screen for a target gene having enhanced functional property. It is also possible to obtain a further improved next generation chimeric DNA library by repeating the inventive process using the first generation chimeric DNA library generated as above as a starting material.

The following Examples and Test Examples are given for the purpose of illustration only, and are not intended to limit the scope of the invention.

### EXAMPLE 1

### Preparation of Template DNA

To test the inventive method, the construction of a chimeric DNA library was carried out through reassembly of heterologous DNA of chitinase genes.

The total nucleic acid of each of Aeromonas hydrophila (KCTC 2358), Pantoea agglomerans (KCTC 2578) and Aeromonas punctata (KCTC 2944) was purified using a genome DNA prep kit (Promega). Each chitinase gene was amplified using the purified nucleic acid as a template and Chi600f of SEQ ID NO. 1 and Chi1200r of SEQ ID NO. 2 as a primer pair. PCR was performed by using Vent polymerase (NEB) and Taq polymerase (Bioneer) together. The reaction procedure consisted of a primary denaturation step at 94° C. for 3 min, 30 cycles of a denaturation step at 94° C. for 30 sec, an annealing step at 50° C. for 30 sec, an extension step at 72° C. for 30 sec, and an further extension step at 72° C. for 30 min. Amplified PCR product was cloned into T-vector (Promega) and subjected to sequence analysis.

The result of sequence analysis showed that chitinase DNAs isolated from Aeromonas hydrophila, Pantoea agglomerans and Aeromonas punctata had nucleotide sequences of SEQ ID NO. 3, NO. 4 and NO. 5, respectively. Their sequence similarity was examined and described in Table 1. Values in the left lower part of the table represent percent similarity, and those in the right upper part, number of different nucleotide sequence/total number of nucleotide sequence.

TABLE 1

| | Pantoea | Aeromonas | Aeromonas |
|---|---|---|---|
| | agglomerans | hydrophila | punctata |
| Pantoea | — | 31/579 | 126/579 |
| agglomerans | | | |
| Aeromonas | 94.65% | — | 124/579 |
| hydrophila | | | |
| Aeromonas | 78.24% | 78.58% | — |
| punctata | | | |

As illustrated in Table 1, chitinase genes of Pantoea agglomerans and Aeromonas hydrophila shows a higher sequence similarity of about 95%.

### EXAMPLE 2

### Reassembly Experiment 1

(2-1) Hybridization of Heterologous DNA Sequences

Heterologous DNA sequences used for a reassembly experiment were prepared by digesting a plasmid containing Pantoea agglomerans (KCTC 2578) or Aeromonas hydrophila (KCTC 2358) chitinase gene with restriction enzymes SacII and SpeI and extracting a chitinase gene region from agarose gel. 0.2 μg each of the DNAs were mixed with S1 nuclease buffer (30 mM sodium acetate, 1 mM zinc acetate, 5% (v/v) glycerol) and 300 mM NaCl to a final volume of 30 μl, incubated at 95° C. for 10 min, and then, gradually cooled to induce DNA hybridization.

(2-2) Preparation of Internal Primers

S1 nuclease was added to the reaction mixture of Example (2-1) to a final concentration of 1,000 units/μl, and incubated at 45° C. for 1 hour, to cleave single strand DNA regions which did not form complementary bonds. Double strand DNA fragments formed between matching sequence regions of the two heterologous chitinase genes were extracted from the reaction mixture using phenol:chloroform, recovered by adding ethanol, and dissolved in 15 μl of distilled water. The DNA fragments thus obtained from two heterologous DNA sequences were used as internal primers for DNA reassembly.

(2-3) DNA Reassembly Using Internal Primers

A PCR reaction was performed by using 1 ng each of Pantoea agglomerans (KCTC 2578) and Aeromonas hydrophila (KCTC 2358) chitinase genes prepared in Example 1, 0.1 pmole each of Chi600f (SEQ ID NO. 1) and Chi1200r (SEQ ID NO. 2) primers, and 5 μl of internal primers generated in Example (2-2). The reaction sequence consisted of a primary denaturation step at 94° C. for 3 min, 45 cycles of a denaturation step at 94° C. for 30 sec, an annealing step at 50° C. for 30 sec, an extension step at 72° C. for 5 sec, and a further extension step at 72° C. for 30 min.

(2-4) Amplification of Reassembled DNA Using Terminal Primers

PCR amplification was performed by further adding 25 pmole each of Chi600f (SEQ ID NO. 1) and Chi1200r (SEQ ID NO. 2) primers to the reaction mixture of Example (2-3). The reaction sequence consisted of a primary denaturation step at 94° C. for 3 min, 30 cycles of a denaturation step at 94° C. for 30 sec, an annealing step at 50° C. for 30 sec, an extension step at 72° C. for 30 sec, and a further extension step at 72° C. for 30 min. The amplified PCR product obtained by the above two-step PCR reactions was subjected to agarose gel electrophoresis, DNA fragments of about 600 bp were extracted from agarose gel, cloned into T-vector (Promega), and transformed E. coli DH5α therewith.

(2-5) Confirmation of Reassembled DNA

5 clones were randomly selected among transformed clones, and subjected to chitinase gene amplification using Chi600f primer of SEQ ID NO. 1 and Chi1200r primer of SEQ ID NO. 2. The nucleotide sequence of each amplified DNA fragment was determined by using the same primer pair. Among them, DNA reassembly was found in 2 clones (clone-A1 and A2). Each sequence of clone-A1 and A2 was compared with those of Pantoea agglomerans (KCTC 2578) and Aeromonas hydrophila (KCTC 2358) in the enzyme-digested form (FIGS. 3 and 4). Clone-A1 and A2 comprised the nucleotide sequences of SEQ ID NO. 6 and SEQ ID NO. 7, respectively.

In FIGS. 3 and 4, the dotted portion of nucleotide sequence means sequence equal to that of the standard. In case of clone-A1, a single substitution mutation (C→T) occurred at the 52^{nd} nucleotide, and a single deletion mutation, at the 337^{th} nucleotide. It has been also confirmed that heterologous DNA sequences were reassembled by template switching between the 244^{th} and 269^{th} nucleotides. In case of clone-A2, it has been confirmed that heterologous DNA sequences were reassembled by two template switching events between the 244^{th} and 269^{th} nucleotides and between the 307^{th} and 348^{th} nucleotides, respectively. However, it is obscure whether DNA reassembly at the 141^{st} nucleotide resulted from a single substitution mutation or template switching.

### EXAMPLE 3

### Use of Restriction Enzyme-Digested DNA for Hybridization Reaction (Reassembly Experiment 2)

(3-1) Hybridization of Heterologous DNA Sequences and Preparation of Internal Primers

Chitinase genes of Pantoea agglomerans (KCTC 2578) and Aeromonas hydrophila (KCTC 2358) prepared in Example 1 were subjected to amplify using Chi600f primer of SEQ ID NO. 1 and Chi1200r primer of SEQ ID NO. 2. 0.5 μg each of the chitinase genes were mixed with S1 nuclease buffer (30 mM sodium acetate, 1 mM zinc acetate, 5% (v/v) glycerol) and 300 mM NaCl to a final volume of 20 μl, incubated at 95° C. for 10 min, and gradually cooled to induce DNA hybridization.

Employed in the above procedure were full-length Aeromonas hydrophila DNA amplified by using Chi600f and Chi1200r primer pair, and HpaII-digested DNA fragment of Pantoea agglomerans gene.

The preparation of complementary internal primers by treating with S1 nuclease was performed as in Example (2-2).

(3-2) DNA Reassembly Using Internal Primers and Amplification of Reassembled DNA Using a Large Quantity of Terminal Primers

A PCR reaction was performed using 1 ng each of Pantoea agglomerans (KCTC 2578) and Aeromonas hydrophila (KCTC 2358) gene prepared in Example 1, 0.1 pmole each of Chi600f (SEQ ID NO. 1) and Chi1200r (SEQ ID NO. 2) primer, and 4 μl of complementary internal primers generated in Example (2-2). The reaction sequence consisted of a primary denaturation step at 94° C. for 3 min, 45 cycles of a denaturation step at 94° C. for 30 sec, an annealing step at 50° C. for 30 sec, an extension step at 72° C. for 20 sec, and a further extension step at 72° C. for 30 min. The reaction mixture was amplified by the same method as described in Example (2-4), amplified PCR fragment of about 600 bp were cloned into T-vector (Promega), and transformed E. coli DH5α therewith.

(3-3) Confirmation of Reassembled DNA

6 clones were randomly selected among transformed clones, and subjected to chitinase gene amplification by using Chi600f primer of SEQ ID NO. 1 and Chi1200r primer of SEQ ID NO. 2. The nucleotide sequence of each amplified DNA fragment was determined by using the same primer pair.

DNA reassembly was found in 4 of the 6 clones. In clone-B62 and B-67, in particular, reassembly occurred by template switching between the 72^{nd} and 147^{th} nucleotides (FIGS. 5 and 6). These two clones showed template switching at the same region, but single nucleotide substitution took place at two sites in clone-B62 and while at one single site in clone-B67. Clone-B63 and B69 exhibited template switching between the 72^{nd} and 147^{th} nucleotides, and in case of clone-B63, further template switching took place between the 210^{th} and 225^{th} nucleotides (FIGS. 7 and 8). In case of clone-B69, further template switching occurred between the 312^{nd} and 351^{th} nucleotides. Clone-B62, B67, B63 and B69 comprised the nucleotide sequence described in SEQ ID NO. 8, NO. 9, NO. 10 and NO. 11, respectively.

### EXAMPLE 4

### 2-Step Hybridization by Rapid Cooling Denaturation and 65° C. Reaction (Reassembly Experiment 3)

(4-1) 2-Step Hybridization of Heterologous DNA Sequences and Preparation of Internal Primer

0.5 μg each of Pantoea agglomerans (KCTC 2578) and Aeromonas hydrophila (KCTC 2358) chitinase DNA were mixed, heated at 95° C for 10 min, and then, incubated in ice to denature DNA. S1 nuclease buffer (30 mM sodium acetate, 1 mM zinc acetate, 5% (v/v) glycerol) and 300 mM NaCl were added thereto, and reacted at 65° C. for 2 hours to induce hybridization.

The recovery of complementary internal primers by treating with S1 nuclease was performed as in Example (2-2).

(4-2) DNA Reassembly Using Internal Primers and Amplification of Reassembled DNA Using a Large Quantity of Terminal Primers

PCR reaction was performed by using 1 ng each of Pantoea agglomerans (KCTC 2578) and Aeromonas hydrophila (KCTC 2358) prepared in Example 1, 0.1 pmole each of Chi600f (SEQ ID NO. 1) and Chi1200r (SEQ ID NO. 2) primer, and 4 μl of complementary internal primers generated in Example (4-1). The reaction sequence consisted of a primary denaturation step at 94° C for 3 min, 45 cycles of a denaturation step at 94° C. for 30 sec, an annealing step at 50° C. for 30 sec, an extension step at 72° C. for 20 sec, and a further extension step at 72° C. for 30 min. 25 pmole each of Chi600f primer of SEQ ID NO. 1 and Chi1200r primer of SEQ ID NO. 2 were further added to the reaction mixture, and the reaction mixture was subjected to amplification. The PCR reaction sequence consisted of a primary denaturation step at 94° C. for 3 min, 30 cycles of a denaturation step at 94° C. for 30 sec, an annealing step at 50° C. for 30 sec and an extension step at 72° C. for 20 sec, and a further extension step at 72° C. at 30 min. PCR fragments of about 600 bp thus amplified was cloned into T-vector (Promega), and transformed into E. coli DH5α therewith.

(4-3) Confirmation of Reassembled DNA

6 clones were randomly selected among transformed clones, and subjected to a chitinase gene amplification by using Chi600f primer of SEQ ID NO. 1 and Chi1200r primer of SEQ ID NO. 2. The nucleotide sequence of each amplified DNA fragment was determined using the same primer pair.

DNA reassembly was found in 3 of 6 clones. In clone-B28, template switching was observed at three sites between the 273^{rd} and 306^{th} nucleotides, between the 351^{st} and 360^{th} nucleotides, and between the 417^{th} and 430^{th} nucleotides, respectively (FIG. 9). In case of clone-B29, template switching took place at four times between the 147^{th} and 207^{th} nucleotides, between the 312^{nd} and 351^{st} nucleotides, between the 363^{rd} and 399^{th} nucleotides, and between the 430^{th} and 450^{th} nucleotides, respectively (FIG. 10). A single template switching between the 312^{nd} and 351^{st} nucleotides occurred in clone-B32 (FIG. 11). Clone-B28, B29 and B32 comprised the nucleotide sequences described in SEQ ID NO. 12, NO. 13 and NO. 14, respectively.

### EXAMPLE 5

### Use of DNA Having Cleaved Ends as a Template (Reassembly Experiment 4)

If an uncleaved, full-length of DNA is used, such DNA is amplified simultaneously with reassembled DNAs during the 2^{nd} step PCR reaction, with a consequential reduction in the amount of reassembled DNAs. To solve this problem, a DNA having its both ends cleaved to remove binding sites of terminal primers is employed as a template in a PCR reaction.

(5- 1) Preparation of Reassembled DNA

Chitinase genes of Pantoea agglomerans (KCTC 2578) and Aeromonas hydrophila (KCTC 2358) prepared in Example 1 were amplified using Chi600f primer of SEQ ID NO. 1 and Chi1200r primer of SEQ ID NO. 2. 2.5 μg each of Pantoea agglomerans (KCTC 2578) and Aeromonas hydrophila (KCTC 2358) chitinase DNA were mixed with S1 nuclease buffer (30 mM sodium acetate, 1 mM zinc acetate, 5% (v/v) glycerol) and 300 mM NaCl to a final volume of 50 μl. The mixture was reacted at 95° C. for 10 min, and gradually cooled to induce DNA hybridization. The recovery of complementary internal primers by treating with S1 nuclease was performed as in Example (2-2), and the recovered DNA fragment was dissolved in 20 μl of distilled water.

Each of the above chitinase gene DNAs was digested with BglII and HinfI to cleave both the 5′- and 3′-ends thereof. A PCR reaction was performed by adding 1 ng each of the enzyme-digested chitinase genes, 0.1 pmole of each Chi600f (SEQ ID NO. 1) and Chi1200r (SEQ ID NO. 2) primer, and 2 μl of each complementary internal primer generated by S1 nuclease. The reaction sequence consisted of a primary denaturation at 94° C. for 3 min, 45 cycles of a denaturation step at 94° C. for 30 sec, an annealing step at 50° C. for 30 sec, an extension step at 72° C. for 20 sec, and a further extension step at 72° C. for 30 min. PCR fragments of about 600 bp amplified by the same method as in Example (2-4) was cloned into T-vector (Promega), and transformed E. coli DH5α therewith.

(5-2) Confirmation of Reassembled DNA

6 clones were randomly selected from the transformed clones, and subjected to chitinase gene by using Chi600f primer of SEQ ID NO. 1 and Chi1200r primer of SEQ ID NO. 2. The nucleotide sequence of each amplified DNA fragments was determined by using the same primer pair.

DNA reassembly was found in 3 of 6 clones. Clone-B1 had a template switching between the 222^{nd} and 229^{th} nucleotides (FIG. 12), and clone-B5, between the 60^{th} and 66^{th} nucleotides (FIG. 13). In case of clone-B6, template switching took place at two sites between the 33^{rd} and 47^{th} nucleotides and between the 428^{nd} and 445^{th} nucleotides, respectively (FIG. 14). Clone-B1 and B6 had the nucleotide sequences described in SEQ ID NO. 15 and NO. 16, respectively.

### EXAMPLE 6

### Reassembly Between Heterologous DNA Sequences Having a Low Sequence Similarity (Reassembly Experiment 5)

The proceeding Examples showed that the construction of a reassembled DNA library from heterologous DNA sequences having 94% sequence similarity could be effectively performed by using the inventive method with high reassembly efficiency. Attempted in this Example was reassembly between heterologous DNA sequences having a relatively low sequence similarity by using the inventive method, and chitinase genes of Pantoea agglomerans (KCTC 2578) and Aeromonas punctata (KCTC 2944) having about 78% sequence similarity were employed.

(6-1) Preparation of Reassembled DNA

Chitinase genes of Pantoea agglomerans (KCTC 2578) and Aeromonas punctata (KCTC 2944) prepared in Example 1 were amplified using Chi600f primer of SEQ ID NO. 1 and Chi1200r primer of SEQ ID NO. 2. 2.5 μg each of Pantoea agglomerans (KCTC 2578) and Aeromonas punctata (KCTC 2944) chitinase DNA were mixed with S1 nuclease buffer (30 mM sodium acetate, 1 mM zinc acetate, 5% (v/v) glycerol) and 300 mM NaCl to a final volume of 50 μl. The mixture was reacted at 95° C. for 10 min, and gradually cooled to induce DNA hybridization. The recovery of complementary internal primers by treating with S1 nuclease was performed as in Example (2-2), and the recovered internal primers were dissolved in 20 μl of distilled water.

Pantoea agglomerans (KCTC 2578) DNA was digested with BglII or HinfI to cleave its 5′-end or 3′-end, and the BglII and HinfI-digested DNAs thus obtained separately were mixed and employed as a template. Also, Aeromonas punctata (KCTC 2944) DNA was digested with DraI or NaeI to obtain 3′-end cleaved or 5′-end cleaved DNA, and the DraI and NaeI-digested DNAs thus obtained were employed as a template.

First, a PCR reaction was performed using 1 ng each of the above two templates, 0.1 pmole each of Chi600f (SEQ ID NO. 1) and Chi1200r (SEQ ID NO. 2) primer, and 2 [l of each complementary internal primer generated by S1 nuclease. The reaction sequence consisted of a primary denaturation step at 94° C. for 3 min, 45 cycles of a denaturation step at 94° C. for 30 sec, an annealing step at 50° C. for 30 sec, an extension step at 72° C. for 20 sec, and a further extension step at 72° C. for 30 min. PCR fragments of about 600 bp amplified with a large quantity of terminal primers by the same method as described in Example (2-4) was cloned into T-vector (Promega), and transformed into E. coli DH5α therewith.

(6-2) Confirmation of Reassembled DNA

6 clones were randomly selected from transformed clones, and subjected to chitinase gene amplification using Chi600f primer of SEQ ID NO. 1 and Chi1200r primer of SEQ ID NO. 2. The nucleotide sequence of each amplified DNA fragments was determined using the same primer pair.

DNA reassembly was found in 1 of 6 clones. Clone-B10 had a template switching between the 327^{th} and 332^{nd} nucleotides, and showed the nucleotide sequence described in SEQ ID NO. 17 (FIG. 15). This result confirms that it is possible to reassemble heterologous DNA sequences having a relatively low sequence similarity.

While the invention has been described with respect to the above specific embodiments, it should be recognized that various modifications and changes might be made to the invention by those skilled in the art which also fall within the scope of the invention as defined by the appended claims.

1. A method for constructing a chimeric DNA library from heterologous DNA sequences, which comprises the steps of:
a) obtaining single strands of the heterologous DNA sequences and hybridizing them to obtain a partially hybridized DNA;
b) cleaving single strand regions of the partially hybridized DNA using a single strand-specific DNase to generate double strand DNA fragments;
c) denaturing the double strand DNA fragments to generate single strand oligonucleotides;
d) conducting a series of polymerase chain reactions (PCR) using the single strand oligonucleotides as internal primers and the single strands obtained in Step (a) as templates to obtain elongated reassembled DNAs; and
e) amplifying the reassembled DNA by PCR using terminal primers which have the nucleotide sequence complementary to those at the both ends of the heterologous DNAs to be reassembled.

2. The method of claim 1, wherein Step (a) is performed by heating a mixture of heterologous DNA sequences to obtain a mixture of single strands thereof, rapidly cooling the mixture, adding a salt thereto, and reacting the mixture at a temperature of 50 to 70° C. for a period of 1 to 12 hours to induce partial hybridization between the single strands of the heterologous DNA sequences.

3. The method of claim 1, wherein Step (a) is performed by treating a mixture of heterologous DNA sequences with a concentrated salt solution to obtain a mixture of single strands thereof and gradually cooling the mixture.

4. The method of claim 1, wherein the single strand-specific DNase is S1 nuclease or Mung Bean nuclease.

5. The method of claim 1, wherein the cleavage reaction in Step (b) is carried out by employing 100 to 1,000 units/ml of the single strand-specific DNase at a temperature ranging from 37 to 45° C.

6. The method of claim 1, wherein terminal primers having nucleotide sequences complementary to both ends of the heterologous DNAs to be reassembled are further added to the PCR reaction mixture in Step (d).

7. The method of claim 6, wherein the terminal primers are added to a concentration ranging from 0.02 to 0.1 pmole/50 μl.

8. The method of claim 1, wherein the concentration of terminal primers used in Step (e) ranges from 20 to 25 pmole/50 μl.

9. The method of claim 1, wherein one of the single strand heterologous DNA sequences in Step (a) is pre-digested with a restriction enzyme.

10. The method of claim 1, wherein the template single strands used in Step (d) are obtained after digesting the heterologous DNA sequences with a restriction enzyme to cleave both ends thereof.

11. The method of claim 1, which further comprises the step of transforming an eukaryotic or prokaryotic cell with the amplified reassembled DNA.

12. The method of claim 1, wherein artificially synthesized primers are used instead of the internal primers generated in Steps (a) to (c).

13. A method of conducting directed evolution which comprises the steps of screening reassembled DNAs from the chimeric DNA library constructed by the method of claim 1 and selecting a DNA having a desired functional property.

14. The method of claim 13, wherein the following steps are repeated using the selected DNA sequence as a starting material:
a) obtaining single strands of the heterologous DNA sequences and hybridizing them to obtain a partially hybridized DNA;
b) cleaving single strand regions of the partially hybridized DNA using a single strand-specific DNase to generate double strand DNA fragments;
c) denaturing the double strand DNA fragments to generate single strand oligonucleotides;
d) conducting a series of polymerase chain reactions (PCR) using the single strand oligonucleotides as internal primers and the single strands obtained in Step (a) as templates to obtain elongated reassembled DNAs; and
e) amplifying the reassembled DNA by PCR using terminal primers which have the nucleotide sequence complementary to those at the both ends of the heterologous DNAs to be reassembled.
